(19) United States
(12) Patent Application Publication
(10) Pub. No.: US 2021/0177585 A1
(43) Pub. Date: Jun. 17, 2021

(54) Prosthetic Heart Valve Tether and Tether Attachment Features

(71) Applicant: Tendyne Holdings, Inc., St. Paul, MN (US)

(72) Inventor: Hendrik J. deHoog, Robbinsdale, MN (US)

(73) Assignee: Tendyne Holdings, Inc., St. Paul, MN (US)

(21) Appl. No.: 17/109,204

(22) Filed: Dec. 2, 2020

Related U.S. Application Data
(60) Provisional application No. 62/948,871, filed on Dec. 17, 2019.

(51) Int. Cl. A61F 2/24

(52) U.S. Cl. CPC A61F 2/2418; A61F 2220/0058; A61F 2230/0023; A61F 2220/0008

(57) ABSTRACT

A prosthetic heart valve may include a collapsible and expandable valve frame, and a prosthetic valve assembly disposed within the valve frame. A tether may extend between a first end and a second end, the second end being coupled to the valve frame. The tether may be formed of a metal filament and have a length sufficient to extend through a ventricular wall when the prosthetic heart valve is implanted in an atrioventricular valve annulus. An anchor system may be provided and may include an epicardial anchor and a locking mechanism to crimp or to otherwise frictionally engage the tether without piercing the tether. Additional accessory tools may be provided to assist in crimping or uncramping the tether from the epicardial pad.

### CROSS-REFERENCE TO RELATED APPLICATIONS

This application claims the benefit of the filing date of U.S. Provisional Patent Application No. 62/948,871, filed Dec. 17, 2019, the disclosure of which is hereby incorporated by reference herein.

### BACKGROUND OF THE DISCLOSURE

Embodiments are described herein that relate to devices and methods for use in the delivery and deployment of prosthetic heart valves, and particularly to tethers and tether attachment features for prosthetic heart valves.

Prosthetic heart valves can pose particular challenges for delivery and deployment within a heart. Valvular heart disease, specifically aortic and mitral valve disease, is a significant health issue in the United States. Traditional valve replacement surgery involving the orthotopic replacement of a heart valve is considered an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure largely due to the necessity for extra-corporeal circulation. Further, open heart surgery is often poorly tolerated in elderly patients. Thus, the elimination of the extra-corporeal component of the procedure could result in a reduction in morbidities and the cost of valve replacement therapies could be significantly reduced.

While replacement of the aortic valve in a transcatheter manner is the subject of intense investigation, less attention has been focused on the mitral valve. This is in part reflective of the greater level of complexity associated with the native mitral valve, and thus a greater level of difficulty with regard to inserting and anchoring the replacement prosthesis. A need therefore exists for delivery devices and methods for transcatheter mitral valve replacement.

Some known delivery methods include delivering a prosthetic mitral valve through an apical puncture site. In such a procedure, the valve is placed in a compressed configuration within a lumen of a delivery catheter of, for example, 34-36 French (Fr) (i.e., an outer diameter of about 11-12 mm). Delivery of a prosthetic valve to the atrium of the heart can be accomplished, for example, via a transfemoral approach, transatrially directly into the left atrium of the heart or via a jugular approach. After the prosthetic heart valve has been deployed, various known anchoring techniques have been used. For example, some prosthetic heart valves are anchored within the heart using anchoring mechanisms attached to the valve, such as barbs, or other features that can engage surrounding tissue in the heart and maintain the prosthetic valve in a desired position within the heart. Some known anchoring techniques include the use of an anchoring tether that is attached to the valve and anchored to a location on the heart, such as an interior or exterior wall of the heart. The present disclosure is generally directed to improvements in such tethers and accessories for tethers.

### BRIEF SUMMARY

According to a first aspect of the disclosure, a prosthetic heart valve includes a collapsible and expandable valve frame, and a prosthetic valve assembly disposed within the valve frame. A tether extends between a first end and a second end. The second end of the tether is coupled to the valve frame. The tether may be formed of a metal filament and have a length sufficient to extend through a ventricular wall when the prosthetic heart valve is implanted in an atrioventricular valve annulus.

According to another aspect of the disclosure, an anchor system is for securing a tether of a prosthetic heart valve. The anchor system may include an epicardial anchor and a locking mechanism. The epicardial anchor may have a tether attachment member defining a tether passageway therethrough. The locking mechanism may be positioned within a recess of the tether attachment member. The locking mechanism may have a leading tip and may be movable from a first position in which the leading tip does not intersect the tether passageway to a second position in which the leading tip intersects the tether passageway. The leading tip of the locking mechanism may be configured to frictionally engage the tether, without piercing the tether, when the tether passes through the tether passageway and the locking mechanism is in the second position.

### BRIEF DESCRIPTION OF THE DRAWINGS

FIG. 1 is a schematic illustration of a prosthetic heart valve, according to an embodiment.

FIG. 2 is a schematic illustration of the prosthetic heart valve of FIG. 1 shown disposed within a heart.

FIGS. 3-5 are front, bottom, and top views, respectively, of a prosthetic heart valve according to an embodiment.

FIG. 6 is an opened and flattened view of the inner frame of the prosthetic heart valve of FIGS. 3-5, in an unexpanded configuration.

FIGS. 7 and 8 are side and bottom views, respectively, of the inner frame of FIG. 6 in an expanded configuration.

FIG. 9 is an opened and flattened view of the outer frame of the prosthetic heart valve of FIGS. 3-5, in an unexpanded configuration.

FIGS. 10 and 11 are side and top views, respectively, of the outer frame of FIG. 9 in an expanded configuration.

FIG. 12-14 are side, front, and top views, respectively, of an assembly of the inner frame of FIGS. 6-8 and the outer frame of FIGS. 9-11.

FIG. 15 is a side perspective view of an assembly of an inner frame and an outer frame shown in a biased expanded configuration, according to an embodiment.

FIG. 16 is a side view of the assembly of FIG. 15 with the outer frame shown inverted.

FIG. 17 is a side view of the assembly of FIG. 16 shown in a collapsed configuration within a lumen of a delivery sheath.

FIG. 18 is a side view of the assembly of FIG. 17 shown in a first partially deployed configuration.

FIG. 19 is a side view of the assembly of FIG. 17 shown in a second partially deployed configuration.

FIG. 20 is a side view of the assembly of FIG. 17 shown in a third partially deployed configuration in which the inverted outer frame is substantially deployed outside of the delivery sheath.

FIG. 21 is a side view of the assembly of FIG. 17 shown in a fourth partially deployed configuration in which the outer frame has everted and assumed a biased expanded configuration.

FIGS. 22-24 illustrate steps of a portion of a method of delivering the prosthetic valve of FIGS. 15-21 to an atrium of a heart and within the native mitral annulus.

FIG. 25 is a schematic side view of a tether according to an aspect of the disclosure.

FIG. 26 is a schematic side view of an inner frame of a prosthetic heart valve coupled to the tether of FIG. 25.

FIG. 27 is a schematic view of an epicardial anchor device according to an embodiment of the disclosure.

FIG. 28 is a top view of the epicardial anchor device of FIG. 27 in an unlocked condition.

FIG. 29 is a top view of the epicardial anchor device of FIG. 27 in a locked condition.

FIG. 30 illustrates a crimping tool.

FIG. 31 illustrates the crimping tool of FIG. 30 engaged with the epicardial anchor device of FIG. 28.

FIG. 32 illustrates a lock release tool.

FIG. 33 illustrates the crimping tool of FIG. 30 and the lock release tool of FIG. 32 engaged with the epicardial anchor device of FIG. 29.

### DETAILED DESCRIPTION

Apparatus and methods are described herein for prosthetic heart valves, such as prosthetic mitral valves or prosthetic tricuspid valves. In particular, tethers for use in securing a prosthetic heart valve within the native valve annulus are described herein, including the use of metal tethers such as tethers formed from a nickel titanium alloy such as nitinol. Additional accessory devices for use with such tethers, such as epicardial pads with features to lock the tether in a desired position, are also described herein. As used herein, the terms “substantially,” “generally,” “approximately,” and “about” are intended to mean that slight deviations from absolute, for example plus or minus 10%, are included within the scope of the term so modified. When ranges of values are described herein, those ranges are intended to include sub-ranges. For example, a recited range of 1 to 10 includes 2, 5, 7, and other single values, as well as all sub ranges within the range, such as 2 to 6, 3 to 9, 4 to 5, and others.

A prosthetic heart valve can be delivered to a heart of patient using a variety of different approaches for delivering a prosthetic heart valve (e.g., a prosthetic mitral valve). For example, the prosthetic heart valves described herein can be delivered using a transfemoral delivery approach as described in International Patent Application No. PCT/US15/14572 (“the '572 PCT Application”) and International Patent Application No. PCT/US2016/012305 (“the '305 PCT Application”), the disclosures of which are hereby incorporated by reference herein, or via a transatrial approach or a transjugular approach as described in U.S. Patent Application Pub. No. 2017/0079790 (“the '790 Publication”), the disclosure of which is also hereby incorporated by reference herein. The prosthetic valves described herein can also be delivered transapically if desired.

In one example, where the prosthetic heart valve is a prosthetic mitral valve, the valve is placed within a lumen of a delivery sheath in a collapsed configuration. A distal end portion of the delivery sheath can be disposed within the left atrium of the heart, and the prosthetic mitral valve can be moved out of the lumen of the delivery sheath and allowed to move to a biased expanded configuration. The prosthetic mitral valve can then be positioned within the mitral annulus of the heart.

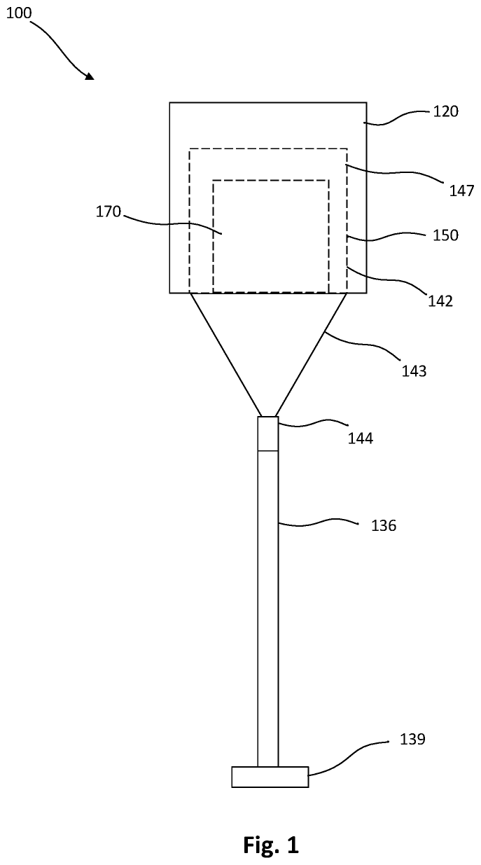

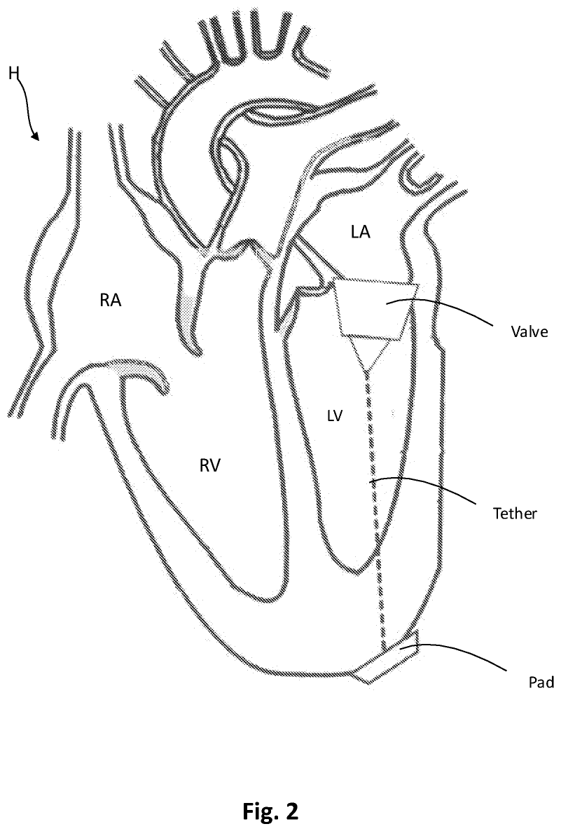

FIG. 1 is a schematic illustration of an example prosthetic heart valve 100. FIG. 2 is a schematic illustration of the example prosthetic heart valve, in this embodiment a prosthetic mitral valve, deployed within a heart H and anchored to a wall of the heart with an epicardial pad via an anchoring tether. The prosthetic heart valve 100 (also referred to herein as “prosthetic valve” or “valve”) can be, for example, a prosthetic mitral valve, although the concepts described herein may apply similarly to a prosthetic tricuspid valve. The valve 100 can be delivered and deployed within an atrium of the heart using a variety of different delivery approaches including, for example, a transapical approach, a transfemoral approach, as described in the '572 PCT Application and the '305 PCT Application, or a transatrial approach or transjugular approach, as described in the '790 Publication.

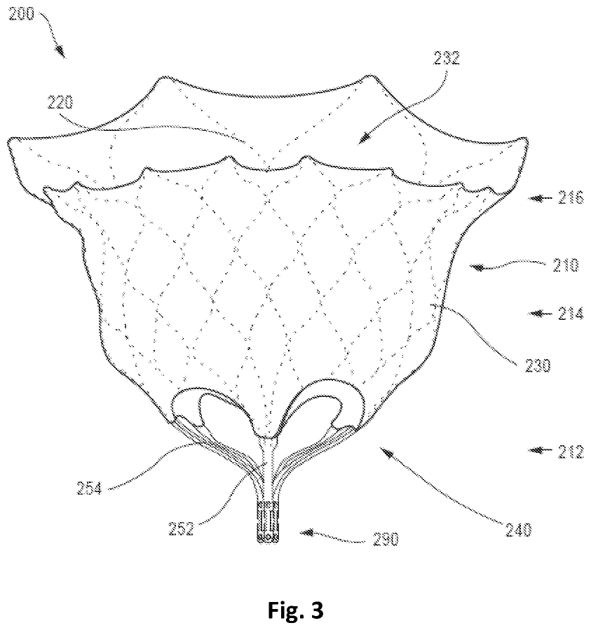

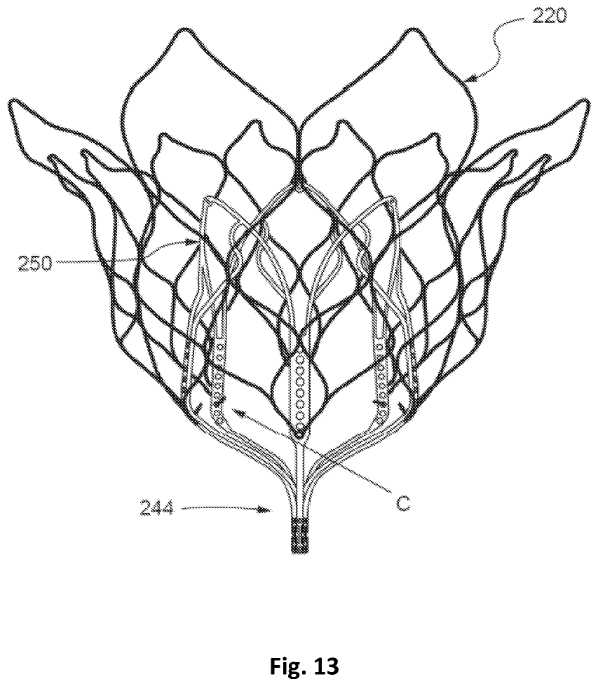

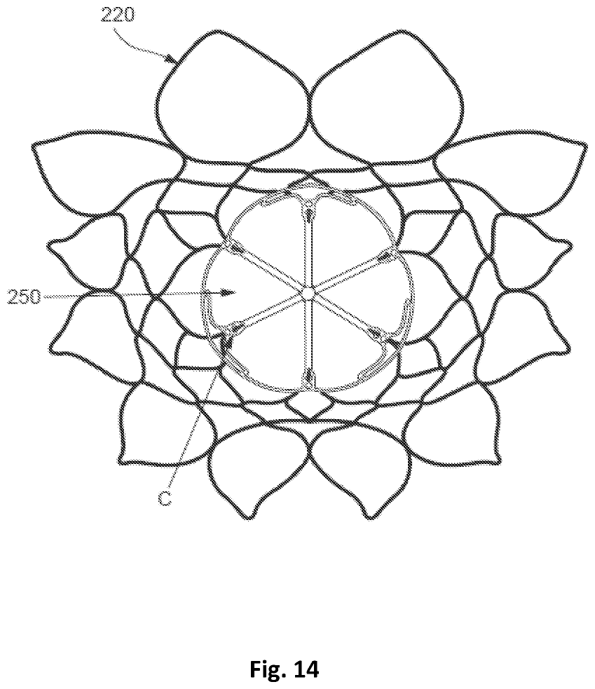

The valve 100 can include an outer frame assembly having an outer frame 120 and an inner valve assembly having an inner frame 150. Each of the outer frame 120 and the inner frame 150 can be formed as a tubular structure as described in more detail below with reference to FIGS. 3-14. The outer frame 120 and the inner frame 150 can be coupled together at multiple coupling joints (not shown) disposed about a perimeter of the inner frame 150 and a perimeter of the outer frame 120. The valve 100 can also include other features, such as those described with respect to FIGS. 3-14 below. For illustration purposes, only the inner frame 150 and the outer frame 120 are discussed with respect to FIG. 1. The various characteristics and features of valve 100 described with respect to FIG. 1 can apply to any of the prosthetic valves described herein.

The outer frame 120 is configured to be biased to an expanded or undeformed shape and can be manipulated and/or deformed (e.g., compressed or constrained) and, when released, return to its original (expanded or undeformed) shape. The inner frame 150 can also be biased to an expanded or undeformed shape and can be manipulated and/or deformed (e.g., compressed and/or constrained) and, when released, return to its original (expanded or undeformed) shape. For example, both the outer frame and the inner frame can be formed of materials, such as metals or plastics, which have shape memory properties. With regard to metals, nickel titanium alloys such as nitinol have been found to be especially useful since they can be processed to be austenitic, martensitic or super elastic. Other shape memory alloys, such as Cu—Zn—Al—Ni alloys, and Cu—Al—Ni alloys, may also be used. Further details regarding the inner frame and the outer frame are described below with respect to valve 200 and FIGS. 3-14.

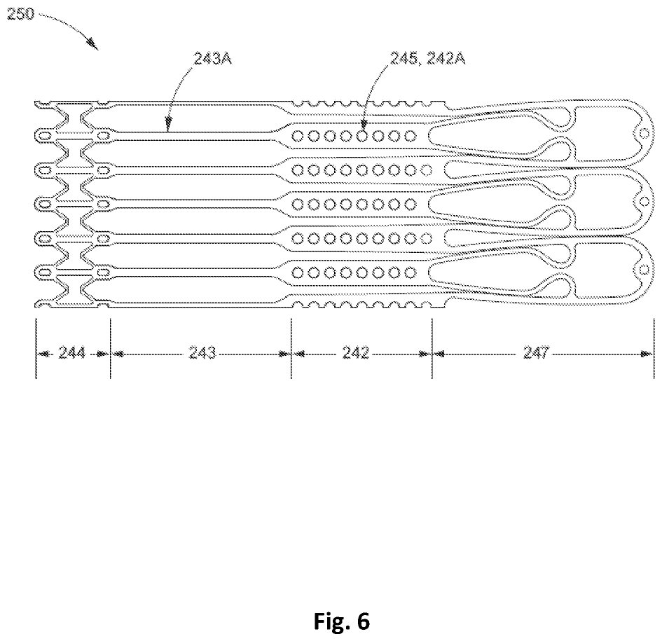

FIGS. 6-8 show an embodiment of an inner frame 250 that is similar to inner frame 150 of FIG. 1. The inner frame 150 can be formed in the same or similar way and include the same or similar portions and/or functions as inner frame 250. The inner frame 150 can be formed from a laser-cut tube of nitinol, and can be divided into four portions corresponding to functionally different portions of the inner frame 150 in final form: atrial portion 147, body portion 142, strut portion 143, and tether clamp or connecting portion 144. In the schematic illustration of FIG. 1, the atrial and body portions (147 and 142) are within the outer frame 120, as indicated by the dashed lines. The valve 100 also includes leaflets 170 (shown in dashed lines) disposed within a portion of the inner frame 150. The leaflets 170 can be formed and configured to be the same as or similar to the leaflets 270 described below with respect to FIGS. 3-14.

The strut portion 143 of inner frame 150 can include a suitable number of individual struts (not shown in FIG. 1 or 2) (see, e.g., struts 243A in FIG. 6) that connect the body portion 142 with the tether connecting portion 144. In some embodiments, the tether connecting portion 144 can include longitudinal extensions of the struts of the strut portion 143 that can be connected circumferentially to one another by pairs of opposed, slightly V-shaped connecting members (or “micro-V's”) (see, e.g., inner frame 250 in FIG. 6). For example, in some embodiments, the strut portion 143 can include six struts that extend to form six struts of the tether connecting portion 144, with each of the six struts of the tether connecting portion 144 being connected circumferentially to one another by micro-V's.

The tether connecting portion or the coupling portion 144 (also referred to as the first end portion of inner frame 150) can be configured to be radially collapsible by application of a compressive force as described in more detail below with reference to valve 200 and inner frame 250. Thus, tether connecting portion 144 can be configured to compressively clamp or grip one end of a tether 136 (e.g., fabric or polymer filament lines braided together), either connecting directly onto the tether 136 or onto an intermediate structure, such as a polymer or metal piece that is in turn firmly fixed to the tether 136. The tether connecting portion 144 can also include openings (not shown in FIG. 1) through which sutures or wires can be inserted to fasten around the collapsed struts and around the end of the tether 136 to couple the tether 136 to the tether connecting portion 144. The tether 136 may be secured to an epicardial pad 139, which in turn may be fixed to an outer surface of the heart. In other embodiments described herein, the tether 136 may be formed of a metal filament instead of a braided fabric, and the tether connecting portion 144 may be correspondingly designed to account for the thinner profile of a metal tether, which may be welded or otherwise coupled to connecting portion 144, for example, instead of being sutured following compressive clamping.

FIGS. 3-14 illustrate another embodiment of a prosthetic heart valve 200. FIGS. 3-5 are front, bottom, and top views, respectively, of prosthetic heart valve 200. The prosthetic heart valve 200 can be delivered and deployed within the left atrium of a heart using a variety of different delivery approaches including, for example, a transapical delivery approach, a transfemoral delivery approach or a transatrial delivery approach. Prosthetic heart valve 200 (also referred to herein as “valve” or “prosthetic valve”) is designed to replace a damaged or diseased native heart valve such as the mitral valve or a tricuspid valve. Valve 200 includes an outer frame assembly 210 and an inner valve assembly 240 coupled to the outer frame assembly 210.

As shown, outer frame assembly 210 includes an outer frame 220, covered on all or a portion of its outer face with an outer covering 230, and covered on all or a portion of its inner face by an inner covering 232. Outer frame 220 can provide several functions for prosthetic heart valve 200, including serving as the primary structure, as an anchoring mechanism and/or an attachment point for a separate anchoring mechanism to anchor the valve within the native heart valve annulus, as a support to carry inner valve assembly 240, and/or as a seal to inhibit paravalvular leakage between prosthetic heart valve 200 and the native heart valve annulus.

Outer frame 220 is biased to an expanded configuration and can be manipulated and/or deformed (e.g., compressed and/or constrained) and, when released, return to its original unconstrained shape. To achieve this, outer frame 220 can be formed of materials, such as metals or plastics, which have shape memory properties. With regard to metals, nitinol has been found to be especially useful since it can be processed to be austenitic, martensitic or super elastic. Other shape memory alloys, such as Cu—Zn—Al—Ni alloys, and Cu—Al—Ni alloys, may also be used.

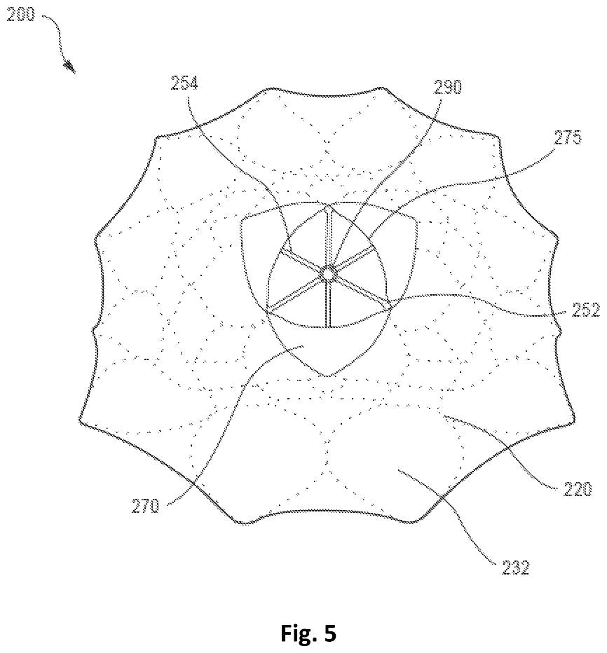

As best shown in FIG. 3, outer frame assembly 210 has an upper end (e.g., at the atrium portion 216), a lower end (e.g., at the ventricle portion 212), and a medial portion (e.g., at the annulus portion 214) therebetween. The upper end or atrium portion 216 (also referred to as “outer free end portion”) defines an open end portion of the outer frame assembly 210. The medial or annulus portion 214 of the outer frame assembly 210 has a perimeter that is configured (e.g., sized, shaped) to fit into the annulus of a native atrioventricular valve. The upper end of the outer frame assembly 210 has a perimeter that is larger than the perimeter of the medial portion. In some embodiments, the perimeter of the upper end of the outer frame assembly 210 has a perimeter that is substantially larger than the perimeter of the medial portion. As shown best in FIG. 5, the upper end and the medial portion of the outer frame assembly 210 have a D-shaped cross-section. In this manner, the outer frame assembly 210 promotes a suitable fit into the annulus of the native atrioventricular valve.

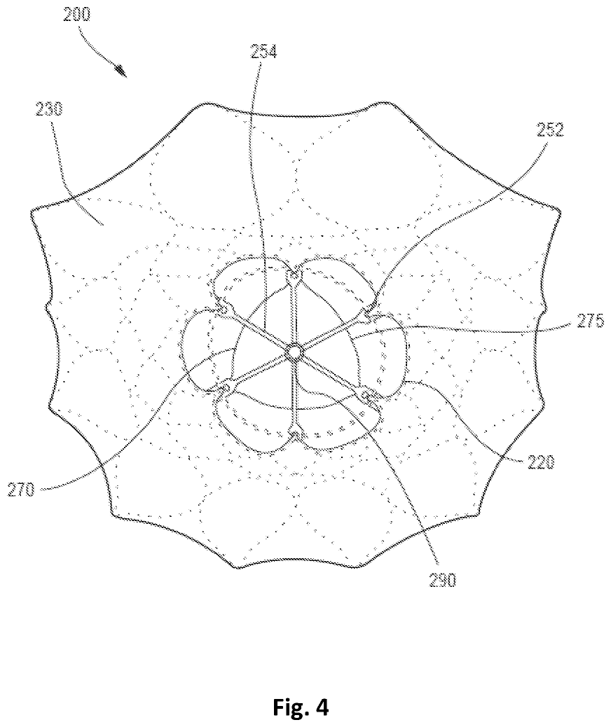

Inner valve assembly 240 includes an inner frame 250, an outer covering (not shown), and leaflets 270. As shown, the inner valve assembly 240 includes an upper portion having a periphery formed with multiple arches. The inner frame 250 may include six axial posts or frame members that support the outer covering of the inner valve assembly and leaflets 270, although a greater or lesser number of such posts is contemplated herein. Leaflets 270 may be attached along three of the posts, shown as commissure posts 252 (best illustrated in FIG. 4), and the outer covering of the inner valve assembly 240 may be attached to the other three posts 254 (best illustrated in FIG. 4), and optionally to commissure posts 252. Each of the outer covering of the inner valve assembly 240 and leaflets 270 is formed of approximately rectangular sheets of material that are joined together at their upper, or atrium end. The lower, ventricle end of the outer covering may be joined to inner covering 232 of outer frame assembly 210, and the lower, ventricle end of leaflets 270 may form free edges 275, though coupled to the lower ends of commissure posts 252.

Although inner valve assembly 240 is shown as having three leaflets, in other embodiments, the inner valve assembly can include any suitable number of leaflets. The leaflets 270 are movable between an open configuration and a closed configuration in which the leaflets 270 coapt, or meet in a sealing abutment.

The outer covering 230 of the outer frame assembly 210, the inner covering 232 of outer frame assembly 210, the outer covering of the inner valve assembly 240, and the leaflets 270 of the inner valve assembly 240 may be formed of any suitable material, or combination of materials, including biocompatible polymers, fabrics, and/or tissue. In this embodiment, the inner covering 232 of the outer frame assembly 210, the outer covering of the inner valve assembly 240, and the leaflets 270 of the inner valve assembly 240 are formed, at least in part, of porcine pericardium. Moreover, in this embodiment, the outer covering 230 of the outer frame assembly 210 is formed, at least in part, of polyester.

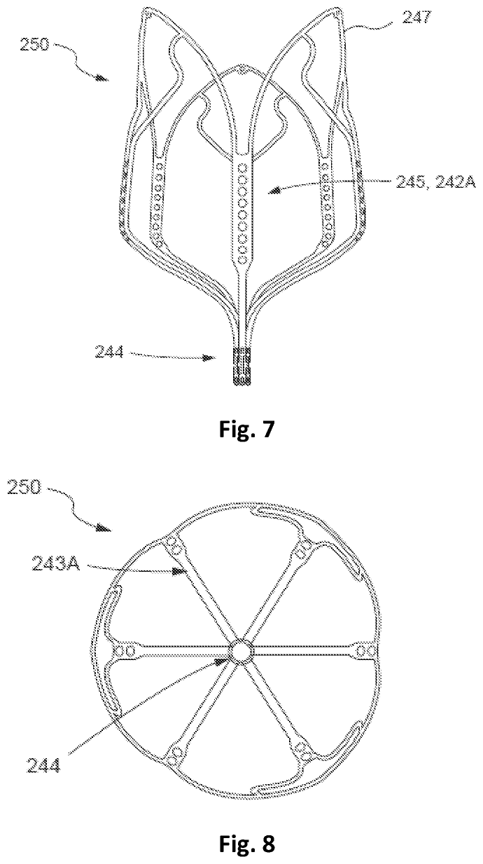

Inner frame 250 is shown in more detail in FIGS. 6-8. Specifically, FIGS. 6-8 show inner frame 250 in an undeformed, initial state (FIG. 6), a side view of the inner frame 250 in a fully deformed, deployed configuration (FIG. 7), and a bottom view of the inner frame 250 in the fully deformed, deployed configuration (FIG. 8), respectively, according to an embodiment.

In this embodiment, inner frame 250 is formed from a laser-cut tube of nitinol. Inner frame 250 is illustrated in FIG. 6 in an undeformed, initial state, e.g. as laser-cut, but cut and unrolled into a flat sheet for ease of illustration. Inner frame 250 can be divided into four portions corresponding to functionally different portions of the inner frame 250 in final form: atrial portion 247, body portion 242, strut portion 243, and tether clamp or connecting portion 244. Strut portion 243 may include six struts, such as strut 243A, which connect body portion 242 to tether connecting portion 244. However, a greater or lesser number of struts is contemplated herein.

Tether connecting portion 244 (also referred to as the first end portion of the inner frame) includes longitudinal extensions of the struts, connected circumferentially to one another by pairs of opposed, slightly V-shaped connecting members (or “micro-V's”). Tether connecting portion 244 is configured to be radially collapsed by application of a compressive force, which causes the micro-V's to become more deeply V-shaped, with the vertices moving closer together longitudinally and the open ends of the V shapes moving closer together circumferentially. Thus, tether connecting portion 244 can be configured to compressively clamp or grip one end of a tether, either connecting directly onto a tether line (e.g., fabric or polymer filament lines braided together) or onto an intermediate structure, such as a polymer or metal piece that is, in turn, firmly fixed to the tether. As noted above and described in greater detail below, if the tether is formed of a thinner material such as a single metal filament, tether connecting portion 244 may be formed with an alternate structure to mate to the metal tether.

In contrast to tether connecting portion 244, atrial portion 247 (also referred to as “inner frame free end portion”) and body portion 242 are configured to be expanded radially. Strut portion 243 forms a longitudinal connection and radial transition between the expanded body portion and the compressed tether connecting portion 244. Body portion 242 provides an inner frame coupling portion 245 that includes six longitudinal posts 242A, although the body portion may include a greater or lesser number of such posts. The inner frame coupling portion 245 can be used to attach leaflets 270 to inner frame 250, and/or can be used to attach inner valve assembly 240 to outer frame assembly 210, such as by connecting inner frame 250 to outer frame 220. In the illustrated embodiment, posts 242A include apertures through which connecting members (such as suture filaments and/or wires) can be passed to couple the posts to other structures.

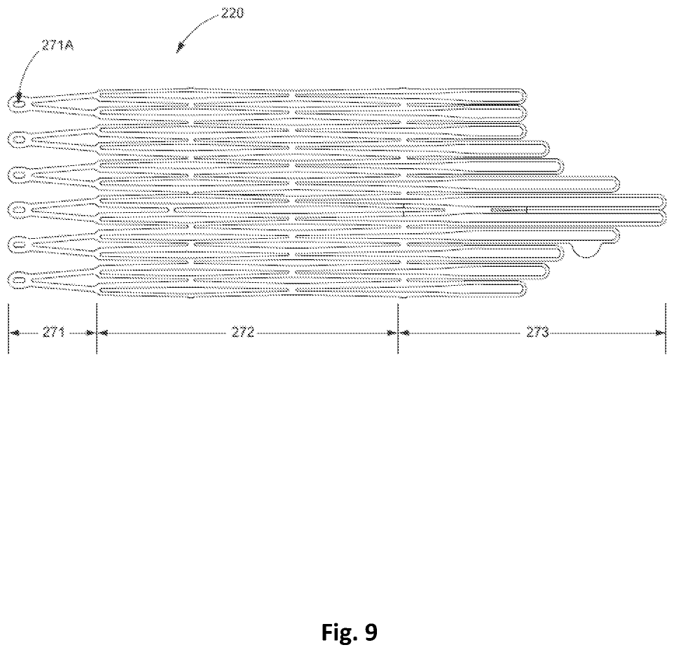

Outer frame 220 of valve 200 is shown in more detail in FIGS. 9-11. In this embodiment, outer frame 220 is also formed from a laser-cut tube of nitinol. Outer frame 220 is illustrated in FIG. 9 in an undeformed, initial state, e.g., as laser-cut, but cut longitudinally and unrolled into a flat sheet for ease of illustration. Outer frame 220 can be divided into an outer frame coupling portion 271, a body portion 272, and a cuff portion 273 (which includes the atrium or free end portion 216), as shown in FIG. 9. Outer frame coupling portion 271 includes multiple openings or apertures 271A, by which outer frame 220 can be coupled to inner frame 250, as discussed in more detail below.

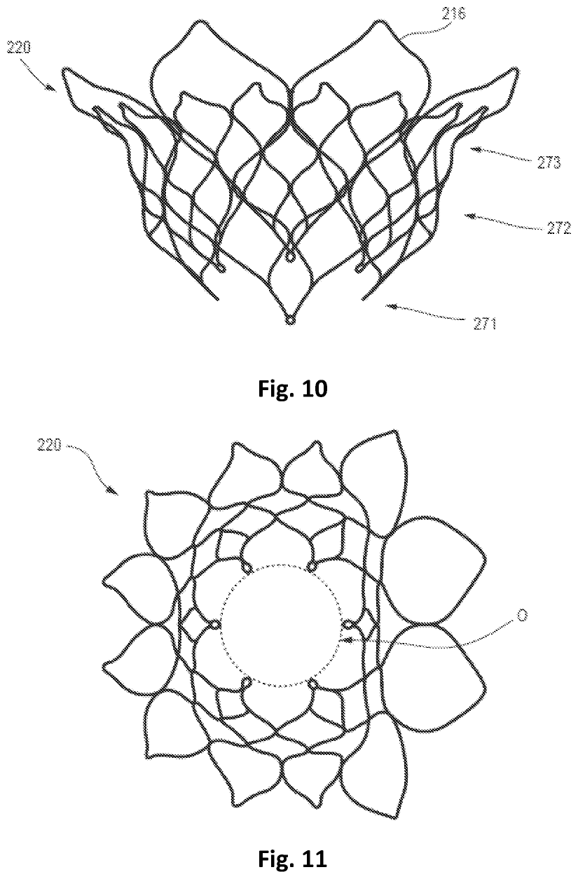

Outer frame 220 is shown fully deformed, e.g., to the final, deployed configuration, in the side view and top view in FIGS. 10 and 11, respectively. As best seen in FIG. 11, the lower end of outer frame coupling portion 271 forms a roughly circular opening (identified by “O” in FIG. 11). The diameter of this opening preferably corresponds approximately to the fully deformed diameter of body portion 242 of inner frame 250, to facilitate the coupling together of these two components of valve 200.

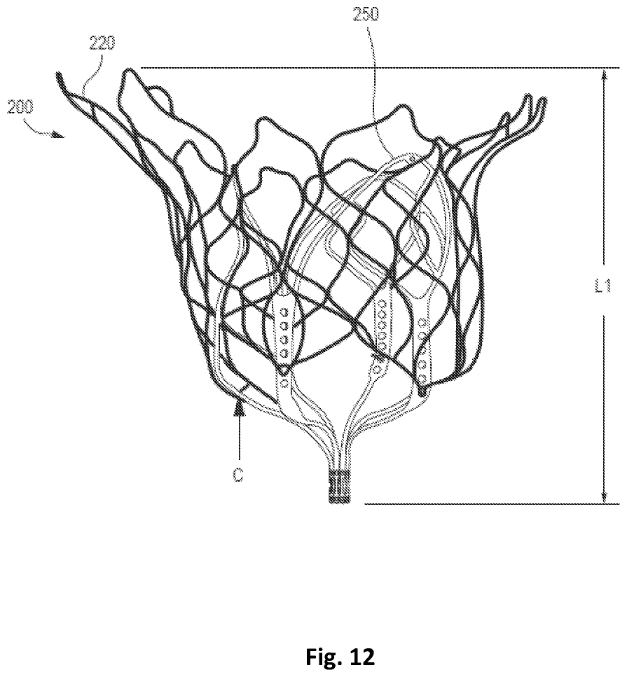

Outer frame 220 and inner frame 250 are shown coupled together in FIGS. 12-14, in front, side, and top views, respectively. The two frames collectively form a structural support for a prosthetic valve, such as valve 200. The frames support the valve leaflet structure (e.g., leaflets 270) in the desired relationship to the native valve annulus, support the coverings (e.g., outer covering 230, inner covering 232, outer covering of inner valve assembly 240) for the two frames to provide a barrier to blood leakage between the atrium and ventricle, and couple to a tether (not shown in FIGS. 3-14) (e.g., tether 136 described above with respect to FIG. 1) to aid in holding the prosthetic valve 200 in place in the native valve annulus by the tether connection to the ventricle wall. The outer frame 220 and the inner frame 250 are connected at six coupling points (representative points are identified as “C” in FIGS. 12-14). In this embodiment, the coupling of the frames is implemented with a mechanical fastener, such as a short length of wire, passed through each aperture 271A in outer frame coupling portion 271 and a corresponding aperture in inner frame coupling portion 245 (e.g., in longitudinal posts 242A) in body portion 242 of inner frame 250. Inner frame 250 is thus disposed within the outer frame 220 and securely coupled to it.

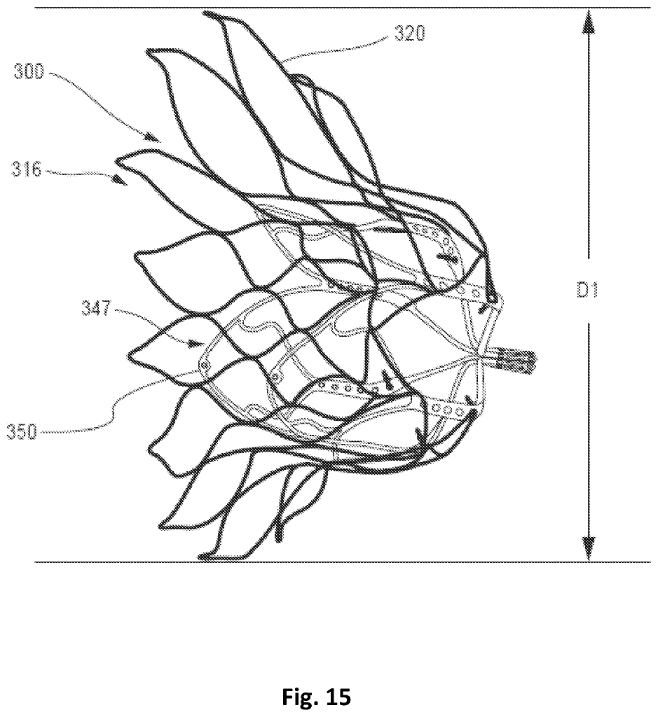

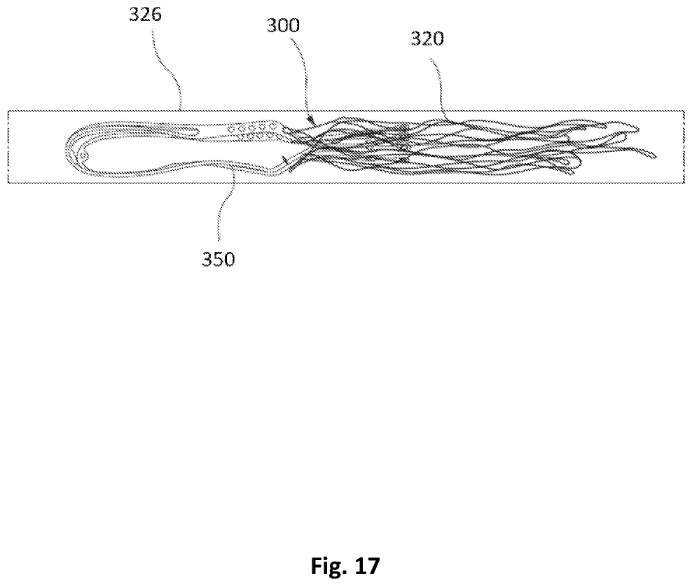

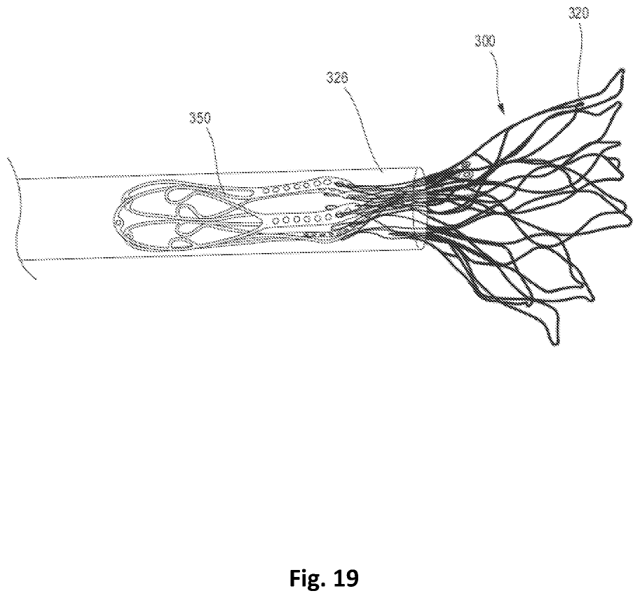

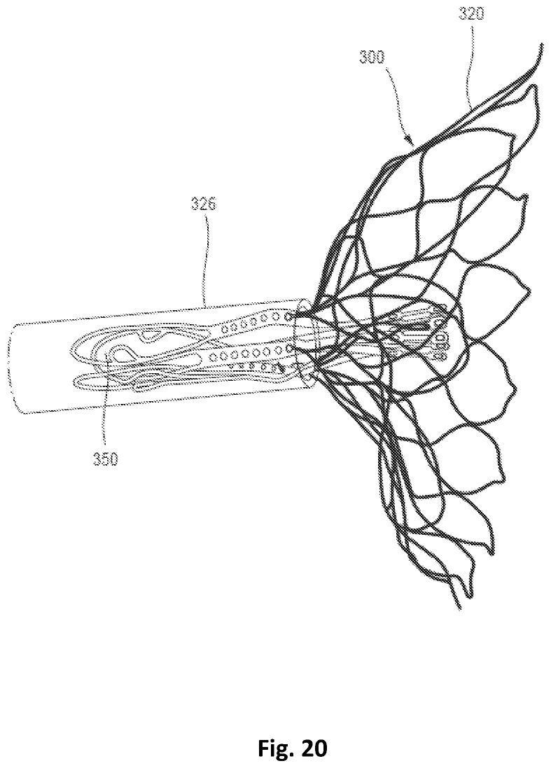

FIGS. 15-21 illustrate a method of reconfiguring a prosthetic heart valve 300 (e.g., prosthetic mitral valve) prior to inserting the prosthetic heart valve 300 into a delivery sheath 326 (see, e.g., FIGS. 17-21) for delivery into the atrium of the heart. The prosthetic heart valve 300 (also referred to herein as “valve”) can be constructed the same as or similar to, and function the same as or similar to, the valves 100 and 200 described above. Thus, some details regarding the valve 300 are not described below. It should be understood that for features and functions not specifically described, those features and functions can be the same as or similar to those of valve 200.

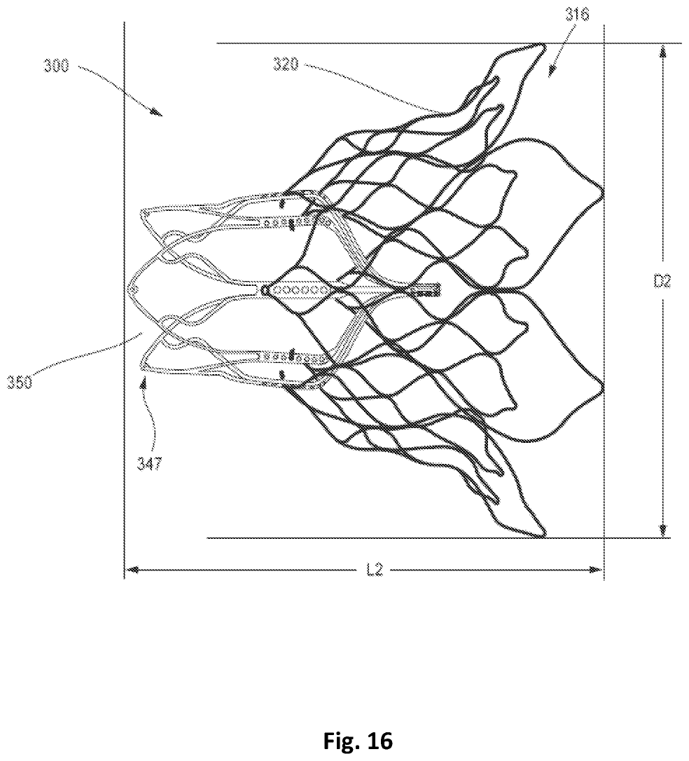

As shown in FIG. 15, the valve 300 has an outer frame 320 and an inner frame 350. As discussed above for valves 100 and 200, the outer frame 320 and the inner frame 350 of valve 300 can each be formed from a shape-memory material and can be biased to an expanded configuration. The outer frame 320 and the inner frame 350 can be moved to a collapsed configuration for delivery of the valve 300 to the heart. In this example method of preparing the valve 300 for delivery to the heart, the outer frame 320 of valve 300 is first disposed in a prolapsed or inverted configuration as shown in FIG. 16. Specifically, the elastic or superelastic structure of the outer frame 320 of valve 300 allows the outer frame 320 to be disposed in the prolapsed or inverted configuration prior to the valve 300 being inserted into the lumen of the delivery sheath 326. As shown in FIG. 16, to dispose the outer frame 320 in the inverted configuration, the outer frame 320 is folded or inverted distally (to the right in FIG. 16) such that an open free end 316 of the outer frame 320 is pointed away from an open free end 347 of the inner frame 350. As described above for valve 100, in this inverted configuration, the overall outer perimeter or outer diameter of the valve 300 is reduced and the overall length is increased. For example, the diameter D1 shown in FIG. 15 is greater than or equal to the diameter D2 shown in FIG. 16, and the length L1 (shown in FIG. 12 for valve 200) is less than the length L2 shown in FIG. 16 for valve 300. With the outer frame 320 in the inverted configuration relative to the inner frame 350, the valve 300 can be placed within a lumen of a delivery sheath 326 as shown in FIG. 17 for delivery of the valve 300 to the left atrium of the heart. By disposing the outer frame 320 in the inverted configuration relative to the inner frame 350, the valve 300 can be collapsed into a smaller overall diameter, e.g. when placed in a smaller diameter delivery sheath, than would be possible if the valve 300 in the configuration shown in FIG. 15 were collapsed radially without being inverted. This is because, in the configuration shown in FIG. 15, the two frames are concentric or nested, and thus the outer frame 320 must be collapsed around the inner frame 350, whereas in the configuration shown in FIG. 16, the two frames are substantially coaxial but not concentric or nested. Thus, in the configuration shown in FIG. 16, the outer frame 320 can be collapsed without the need to accommodate the inner frame 350 inside of it. In other words, with the inner frame 350 disposed mostly inside or nested within the outer frame 320, the layers or bulk of the frame structures cannot be compressed to as small a diameter. In addition, if the frames are nested, the structure is less flexible, and therefore, more force is needed to bend the valve, e.g., to pass through tortuous vasculature or to make a tight turn in the left atrium after passing through the atrial septum to be properly oriented for insertion into the mitral valve annulus.

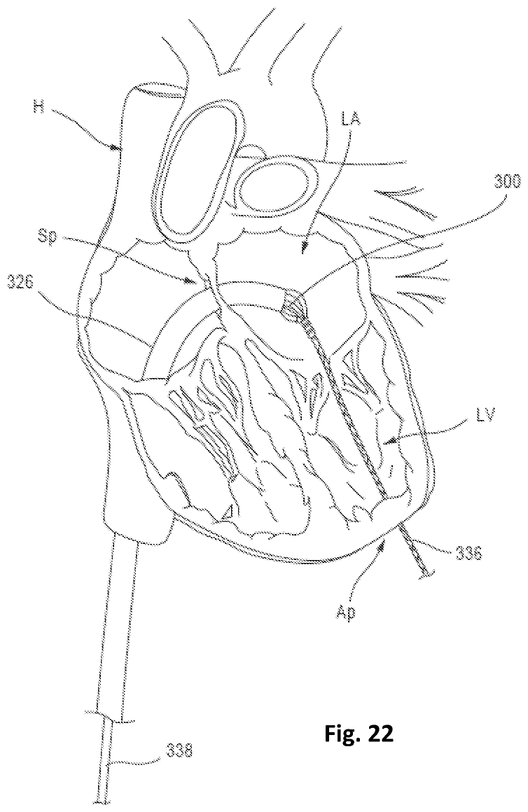

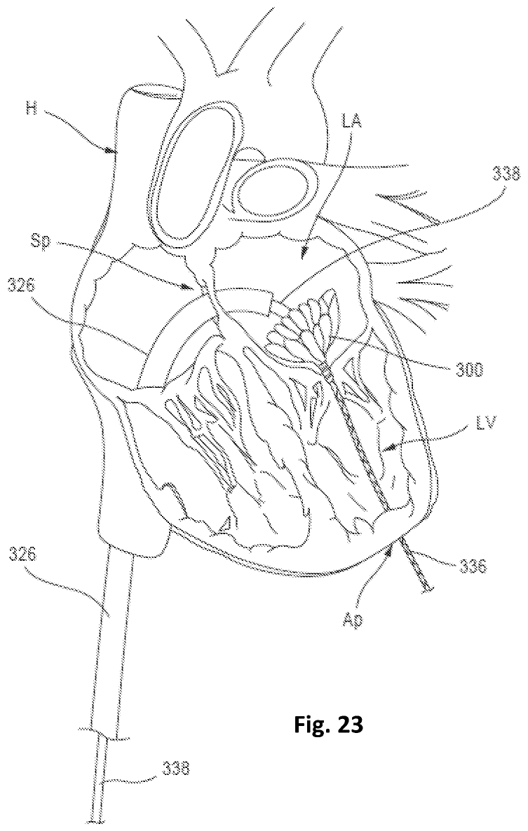

FIGS. 22-24 illustrate a portion of a procedure for delivering the valve 300 to the heart. In this embodiment, the valve 300 is shown being delivered via a transfemoral delivery approach as described, for example, in the '305 PCT Application incorporated by reference above. The delivery sheath 326, with the valve 300 disposed within a lumen of the delivery sheath 326 and in an inverted configuration as shown in FIG. 17, can be inserted into a femoral puncture, through the femoral vein, through the inferior vena cava, into the right atrium, through the septum Sp and into the left atrium LA of the heart. With the distal end portion of the delivery sheath 326 disposed within the left atrium of the heart, the valve 300 can be deployed outside a distal end of the delivery sheath 326. For example, in some embodiments, a pusher device 338 can be used to move or push the valve 300 out from the distal end of the delivery sheath 326. As shown in FIGS. 22-24, a tether 336 can be attached to the valve 300, and extend though the mitral annulus, through the left ventricle LV, and out from the heart through a puncture site at the apex Ap. In some embodiments, the valve 300 can be moved out from the delivery sheath 326 by pulling on the portion of the tether 336 extending out of the patient's chest. In some embodiments, the valve 300 can be deployed by both pushing with the pusher device 338 and pulling with the tether 336.

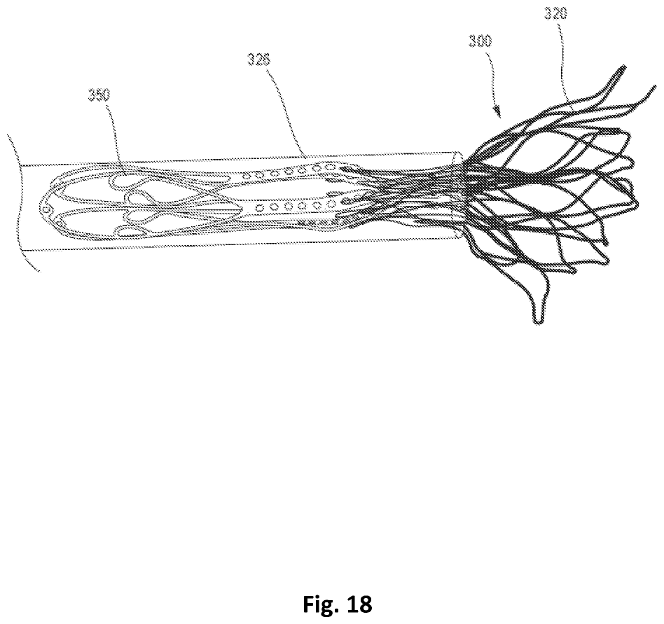

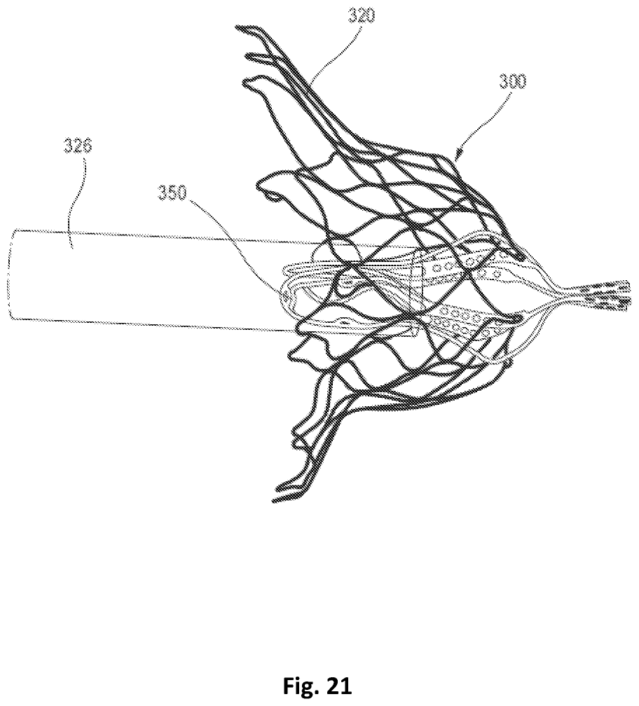

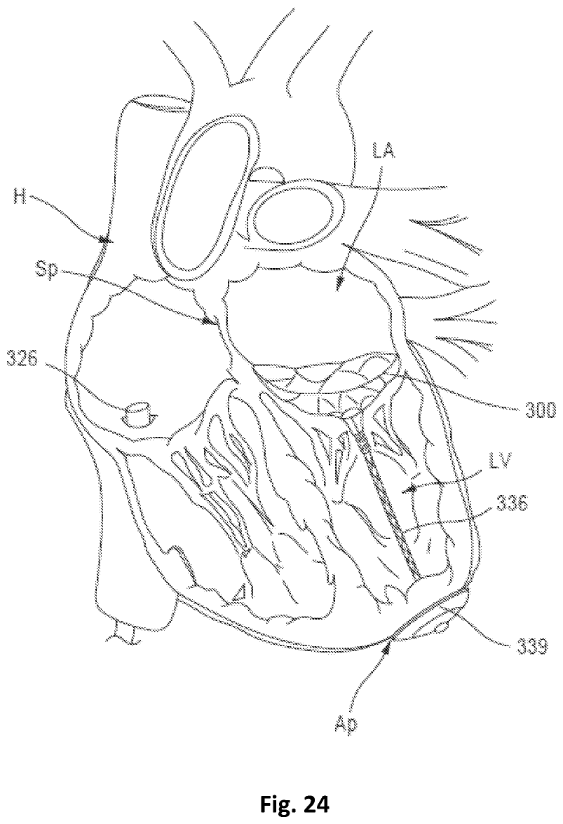

As the valve 300 exits the lumen of the delivery sheath 326, the outer frame assembly 310 exits first in its inverted configuration as shown in the progression of FIGS. 18-20 (see also FIG. 22). After the outer frame assembly 310 is fully outside of the lumen of the delivery sheath 326, the outer frame 320 can revert to its expanded or deployed configuration as shown in FIGS. 21, 23 and 24. In some embodiments, the outer frame 320 can revert automatically after fully exiting the lumen of the delivery sheath due to its shape-memory properties. In some embodiments, a component of the delivery sheath or another device can be used to aid in the reversion of the outer frame assembly 310. In some embodiments, the pusher device 338 and/or the tether 336 can be used to aid in the eversion of the outer frame assembly 310. The valve 300 can continue to be deployed until the inner frame 350 is fully deployed within the left atrium and the valve 300 is in the expanded or deployed configuration (as shown, e.g., in FIGS. 15 and 24). The valve 300 and the tether 336 can then be secured to the apex of the heart with an epicardial pad device 339 as shown in FIG. 24 and as described in more detail in the '572 PCT Application and the '305 PCT Application.

As noted above, the prosthetic heart valves described herein may include a tether in the form of fabric or polymer filament lines braided together, the tether having a first end attached to inner frame 250, for example by compressively clamping tether connection portion 244 over a first end of the tether, with possible additional securement via suturing or other attachment of the first end of the tether to the tether connection portion 244. The second end of the tether may pass partially or completely through the apex Ap of the left ventricle LV, as shown in FIG. 24. The second end of the tether may pass through an aperture in an epicardial pad device, such as epicardial pad 339. The tether may be tensioned to a desired degree, and then the epicardial pad device 339 may be securely coupled to the tether, for example by advancing a pin within the epicardial pad device 339 through the tether 336 to lock the tether at a desired tension. Epicardial pad devices are described in greater detail in U.S. Patent Publication No. 2016/0143736, the disclosure of which is hereby incorporated by reference herein. In other embodiments, described in greater detail below, the tether may be formed of a relatively thin strand of metal, such as a single filament of nitinol, instead of a braided fabric or polymer. While forming the tether from a metal filament may provide a number of benefits, described in greater detail below, such a change may require corresponding changes to one or both of the inner frame 350 and the epicardial pad device 339, also described in greater detail below.

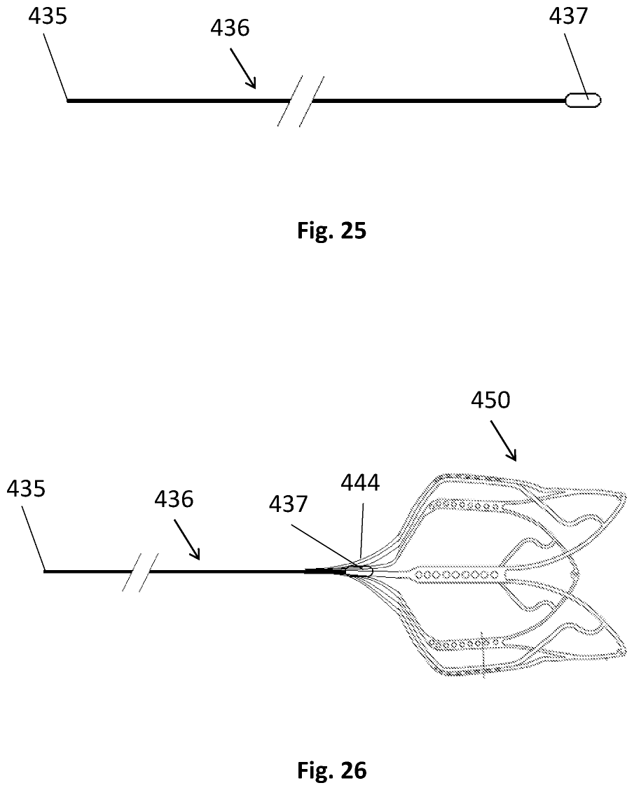

FIG. 25 is a schematic side view of a metal tether 436 that may be used for similar purposes as tether 336. As noted above, tether 436 may be formed of a single filament of nitinol, although other metals or metal alloys may be suitable. It should be understood that the prosthetic heart valve system with which tether 436 is used may be the same as or similar to any of the other prosthetic heart valves described herein, except for certain modifications described below. Thus, the same or similar components of the prosthetic heart valve system with which tether 436 is intended to be used are not described here again. Tether 436 may extend between a first end 435 and a second end 437, and may have a substantially constant width or thickness between the first end 435 and the point at which the tether transitions to second end 437. For example, tether 436 may have a substantially circular cross-section with an outer diameter of about 0.018 inches (0.457 mm). The tether 436 may transition to a plug or bead-shaped second end 437 that has a maximum outer diameter of about 0.030 inches (0.762 mm). It should be understood that these dimensions are merely exemplary. For example, although tether 436 is described as having an outer diameter of about 0.018 inches (0.457 mm), the outer diameter could be smaller or greater, for example between about 0.009 inches (0.223 mm) and about 0.027 inches (0.686 mm). Further, although second end 437 is described as having an outer diameter of about 0.030 inches (0.762 mm), the outer diameter could be smaller or greater, for example between about 0.020 inches (0.051 mm) and about 0.040 inches (1.02 mm). Still further, in some embodiments, while the majority of the tether 436 may have a single outer diameter such as about 0.018 inches (0.223 mm), a length of the tether 436 starting at first end 435 may have a smaller diameter. It is preferable that, whatever the particular dimensions of the majority of tether 436 and second end 437, the second end 437 has a larger diameter than the remainder of the tether 436, such as, for example, a factor of between about 1.5 and about 2.5 times larger.

Whereas the majority of tether 436 may have a relatively small outer diameter, for example of about 0.018 inches (0.457 mm), tethers formed of braided fabric or polymer filaments, such as tether 336, may be substantially larger, having diameters of about 0.055 inches (1.397 mm) for example.

FIG. 26 is a schematic side view of an inner frame 450 coupled to tether 436. Inner frame 450 may be substantially identical to inner frame 250 in most respects other than the tether clamp or connecting portion 444. Tether connecting portion 444, which may also be referred to as a valve stem portion, may be significantly smaller than the tether connecting portion 244 of inner frame 250, at least because the outer diameter of tether 436 is significantly smaller than the outer diameter of the braided fabric or polymer tether used with inner frame 250. Tether connecting portion 444 may have a similar structure as tether connecting portion 244, but is formed so that, when connecting portion 444 is crimped around tether 436, the outer diameter of the connecting portion 444 is significantly smaller than the outer diameter of connecting portion 244. In other embodiments, connecting portion 444 may be provided as struts with free ends (instead of the micro-V structure of tether connecting portion 244), with the struts of the tether connecting portion 444 welded to the tether 436, or swaged or crimped to the tether 436, for example by crimping a hypotube over the free ends of the connecting portion 444 while the tether 436 is positioned between the struts of the tether connecting portion 444. The second end 437 of the tether 436 may be positioned distal to the point or points at which the tether connecting portion 444 is fixed to the tether 436, with the second end 437 helping to ensure that the tether 436 cannot disconnect from the inner frame 450, for example because the outer diameter of the second end 437 is larger than the inner diameter of a hypotube crimped over the free ends of the struts of tether connecting portion 444. Various benefits of the type of connection shown and described in connection with FIG. 26 are described in greater detail below. However, methods and apparatus for coupling the tether 436 to an epicardial pad device are first described immediately below.

As noted above, tethers formed of braided fabric or polymer filaments may be fixed to an epicardial pad, such as epicardial pad 339, by advancing a pin within the epicardial pad through the tether extending through an aperture in the epicardial pad. However, this type of connection mechanism may be difficult or impossible with metal tether 436, at least because it is more difficult to pierce a solid metal filament than a braided fabric or polymer.

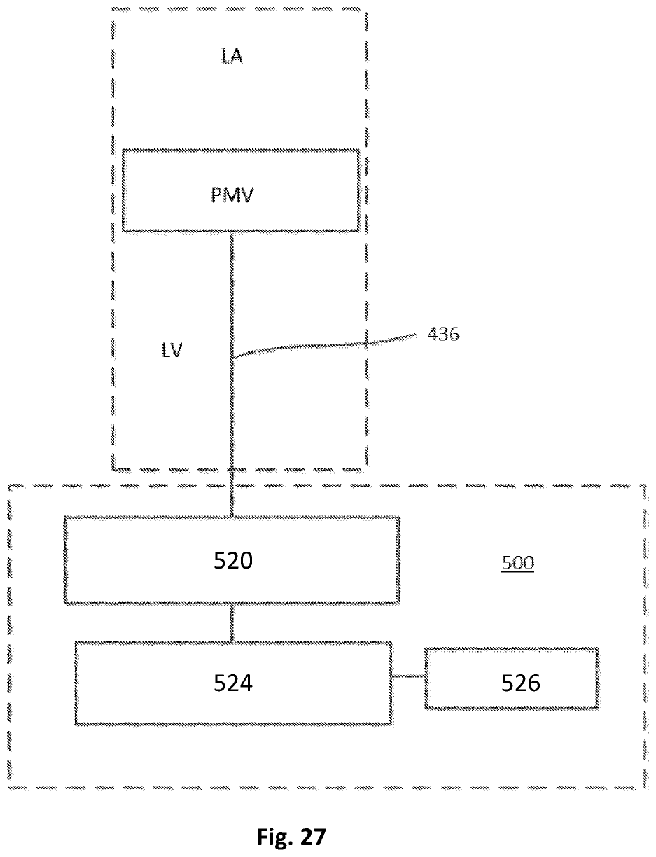

FIG. 27 is a schematic illustration of an epicardial anchor device 500 (also referred to herein as an “epicardial pad,” “anchor device,” or “epicardial anchor”) according to an embodiment of the disclosure. Epicardial anchor device 500 may be used to anchor or secure a prosthetic mitral valve PMV, including one that incorporates inner frame 450, deployed between the left atrium LA and left ventricle LV of a heart. The anchor device 500 can be used, for example, to anchor or secure the prosthetic mitral valve PMV via tether 436, and to seal a puncture formed in the ventricular wall of the heart during implantation of the prosthetic mitral valve PMV. The anchor device 500 can also be used in other applications to anchor a medical device (such as any prosthetic atrioventricular valve or other heart valve) and/or to seal an opening such as a puncture.

The anchor device 500 can include a pad (or pad assembly) 520, a tether attachment member 524 and a locking mechanism 526. The pad 520 can contact the epicardial surface of the heart and can be constructed of any suitable biocompatible surgical material. The pad 520 can be used to assist the sealing of a surgical puncture formed when implanting a prosthetic mitral valve. In some embodiments, the pad 520 can include a slot that extends radially to an edge of the pad 520 such that the pad 520 can be attached to, or disposed about, the tether 436 by sliding the pad 520 onto the tether 436 via the slot. In other embodiments, the pad 520 may include an aperture through which tether 436 may be passed.

In some embodiments, the pad 520 can be made from a double velour material to promote ingrowth into the pad 520 in the puncture site area. For example, pads or pledgets may be made of a felted polyester and may be cut to any suitable size or shape, such as PTFE Felt Pledgets having a nominal thickness of 2.87 mm, available from C. R. Bard, Inc. In some embodiments, the pad 520 can be larger in diameter than the tether attachment member 524. The pad 520 can have a circular or disk shape, or other suitable shapes.

The tether attachment member 524 can provide the anchoring and mounting platform to which one or more tethers 436 can be coupled (e.g., crimped). The tether attachment member 524 can include a base member that defines at least a portion of a tether passageway through which the tether 436 can be received and pass through the tether attachment member 524, and a cavity or recess in which the locking mechanism 526 may be positioned. The locking mechanism cavity may be in fluid communication with the tether passageway such that, when the locking mechanism 526 is disposed in the locking mechanism cavity, the locking mechanism 526 can contact the tether 436 as it passes through the tether passageway as described in more detail below.

The locking mechanism 526 can be used to hold the tether 436 in place after the anchor device 500 has been positioned against the ventricular wall and the tether 436 has been pulled to a desired tension using any suitable mechanism, including manual force, with or without the assistance of a tensioning tool. For example, the tether 436 can extend through a hole in the pad 520 and through the tether passageway of the tether attachment member 524. The locking mechanism 526 can be moved or advanced within the locking mechanism cavity such that it presses against or crimps the tether 436 as the tether 436 extends through the tether passageway of the tether attachment member 524. Thus, the locking mechanism 526 can frictionally engage the tether 436 and secure the tether 436 to the tether attachment member 524.

The tether attachment member 524 can be formed from a variety of suitable biocompatible materials. For example, in some embodiments, the tether attachment member 524 can be made of polyethylene, or other hard or semi-hard polymer, and can be covered with a polyester velour to promote ingrowth. In other embodiments, the tether attachment member 524 can be made of metal, such as, for example, nitinol, or ceramic materials. The tether attachment member 524 can have various sizes and/or shapes. For example, the tether attachment member 524 can be substantially disk shaped.

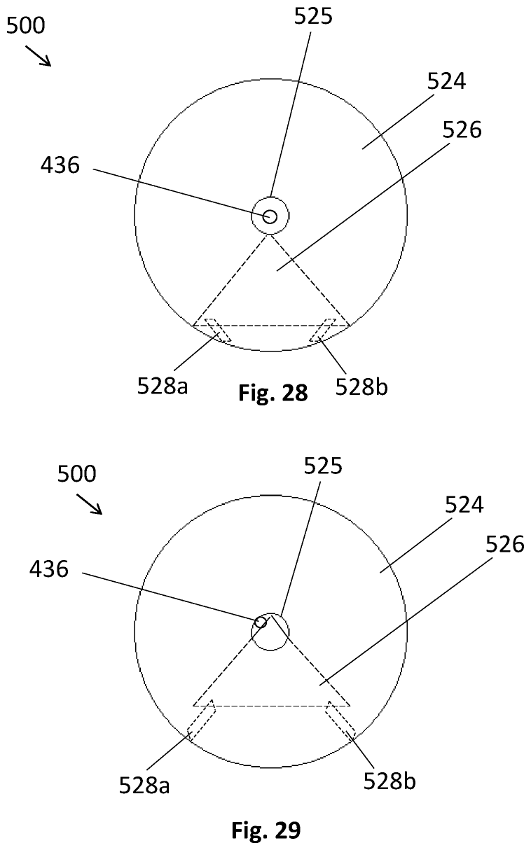

FIG. 28 illustrates anchor device 500 with tether 436 positioned within a tether passageway and the locking mechanism 526 in an unlocked state in which the tether is not secured to the anchor device. FIG. 29 illustrates anchor device 500 with the locking mechanism 526 in a locked state in which the tether 436 is locked and/or crimped. Pad 520 is omitted from FIGS. 28-29 for purposes of clarity. In the illustrated embodiment, tether attachment member 524 is substantially disk shaped and includes a substantially circular interior perimeter 525 that defines, at least in part, the tether passageway through which the tether 436 is adapted to pass. Locking mechanism 526 is illustrated in FIG. 28 in dashed lines to illustrate that the locking mechanism is positioned within an interior cavity of tether attachment member 524, and thus would not otherwise be visible in the illustrated unlocked state. Locking mechanism 526 may have many suitable shapes, and in the illustrated embodiment, the locking mechanism is substantially triangular (e.g., an equilateral or isosceles triangle) and oriented with one of the vertices of the triangle positioned adjacent the interior perimeter 525 in the unlocked state. The cavity in which locking mechanism 526 may be positioned may be formed between a top and bottom portion of the tether attachment member 524, and may for example be a slot having a thickness about the same as, or slightly larger than, the thickness of locking mechanism 526.

In the unlocked state shown in FIG. 28, the leading vertex of locking mechanism 526 is positioned adjacent the interior perimeter 525 so that the leading vertex does not extend into the tether passageway, or only minimally extends into the tether passageway. In the locked state shown in FIG. 29, the locking mechanism 526 has been advanced so that the leading vertex of the locking mechanism has partially or completely traversed the tether passageway, crimping, sandwiching, or otherwise frictionally engaging the tether 436 between the locking mechanism 526 and the interior perimeter 525 of the tether attachment member 524. In the illustrated locked state, the tether 436 is secured so that it cannot move through the tether passageway under typical loads experienced during functioning of the heart and the prosthetic heart valve to which the tether is attached. It should be understood that the locking mechanism may be configured to crimp, sandwich, or otherwise frictionally engage the tether 436 without piercing the tether.

Referring back to FIG. 28, locking mechanism 526 may include one or more position locks 528a-b. In the illustrated embodiment, locking mechanism 526 includes two position locks 528a-b rotatably coupled to the edge of the locking mechanism opposite the leading vertex, to the remaining two vertices of the locking mechanism, or to positions near or adjacent the remaining two vertices. Although two position locks 528a-b are shown, more or fewer may be provided, and the exact positions of the position locks may vary from what is shown. Preferably, position locks 528a-b are biased so that the free or trailing ends of the position locks tend to rotate away from each other. In the view of FIG. 28, position lock 528a is biased to rotate in a clockwise direction, while position lock 528b is biased to rotate in a counterclockwise direction. This rotational bias may be provided by any suitable biasing member, such as a torsion spring. It should be noted, however, that this force alone is insufficient to move the locking mechanism 526 from the unlocked state to the locked state without assistance, as described further below.

In the unlocked state shown in FIG. 28, the trailing ends of position locks 528a-b may lie against an interior surface of the outer perimeter of tether attachment 524, the contact preventing the position locks from rotating. As the locking mechanism 526 is advanced toward the locked state shown in FIG. 29, the position locks 528a-b may become substantially free to rotate due to the rotational bias, resulting in the position shown in FIG. 29. In the locked state, the position locks 528a-b may be wedged between the locking mechanism 526 and the interior surface of the outer perimeter of tether attachment 524. In this position, the position locks 528a-b may support the locking mechanism 526 and prevent or help prevent the locking mechanism 526 from transitioning back to the unlocked state shown in FIG. 28, for example due to forces exerted on the locking mechanism 526 from the crimped tether 436. The interior surface of the outer perimeter of tether attachment 524 may include notches, divots, or other features to assist the position locks 528a-b in maintaining the locked state of the locking mechanism 526. The locking mechanism 526 and position locks 528a-b may be formed of any suitable material, including biocompatible metals, metal alloys, and plastics.

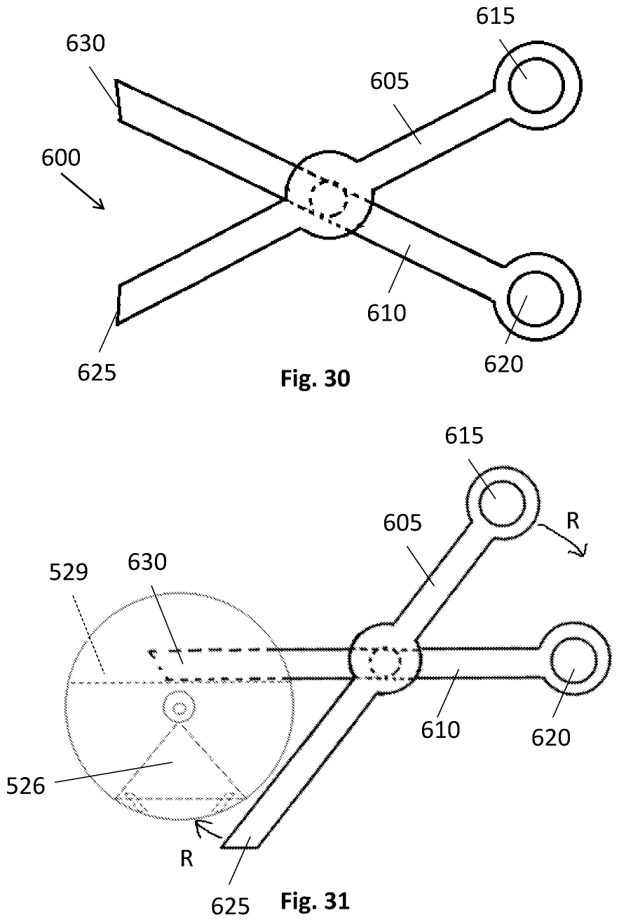

FIG. 30 illustrates an embodiment of a crimping tool 600 that may be used to transition the locking mechanism 526 of anchor device 500 from the unlocked state to the locked state. In the illustrated embodiment, tool 600 takes the general form of a scissor having a first member 605 pivotably coupled to a second member 610. First member 605 and second member 610 may each include a handle end 615, 620, respectively, for a user to grip. First member 605 may terminate in a tip end 625, and second member 610 may terminate in a tip end 630, with the pivot point being between the handle ends 615, 620 and the tip ends, so that a user can maneuver the handle ends to cause the tip ends to rotate toward or away from one another.

As shown in FIG. 31, tether attachment member 524 of anchor device 500 may include an interior wall 529 that may be accessible through a slot formed in the outer perimeter of the tether attachment member 524, for example between a top and a bottom portion of the tether attachment member. The tether 436 and the tether passageway through which the tether extends may be positioned between the interior wall 529 and the locking mechanism 526. The second member 610 of tool 600 may be inserted through that slot until the second member is in abutment with the interior wall 529. Handle end 615 of first member 605 may be rotated in a direction R toward handle end 620, causing the tip end 625 to rotate toward the tip end 630. The tip end 625 may pass through a slot formed in the outer perimeter of the tether attachment member 524 until a portion of first member 605 contacts the edge of the locking mechanism 526 opposite the leading vertex. The first member 605 may avoid contact with the position locks 528a-b, for example if the position locks are coupled to a top or bottom surface of the locking mechanism. In this configuration, the slot opens to the edge of the locking mechanism 526 opposite the leading vertex, while the position locks 528a-b remain in contact with structure on the interior surface of the outer perimeter of the tether attachment member 524. As rotation of handle end 615 continues, the first member 605 of tool 600 will force the locking mechanism 526 to advance toward the tether passageway into the locked state shown in FIG. 29.

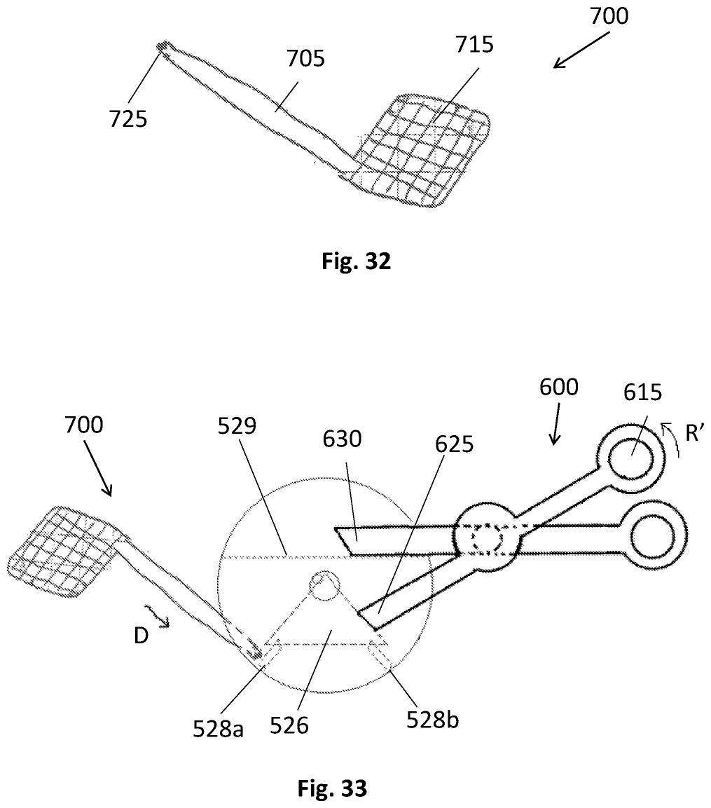

It may also be desirable to allow for the locking mechanism 526 to be transitioned from the locked state back to the unlocked state, for example if it is desired to alter the tension on the tether 436 or to attempt to reposition or remove a prosthetic heart valve that has recently been positioned, but the positioning determined to be undesirable. FIG. 32 illustrates an embodiment of a lock release tool 700. Lock release tool 700 may include a body 705, a handle 715 at one end of the body, and a tip 725 at the other end of the body. Preferably, tip 725 has a shape that is keyed to, or otherwise complementary to, the shape of the position locks 528a-b to assist in engaging the tip of the position lock. Any shape may be suitable, including geometric shapes that provide a large surface area of contact while minimizing profile. The body 705 and/or tip 725 may have a thickness and shape that allows the tip to be inserted into the slot in the tether attachment member 524 adjacent the locking mechanism 526, so that the tip can directly engage the position locks 528a-b. In use, when the anchor device 500 is in the locked state, with the position locks 528a-b in the locked position show in FIG. 29, the tip 725 of the lock release tool 700 may be inserted into the slot and into contact with one of the position locks. The release tool 700 may be used to manually push each position lock 528a-b from the position shown in FIG. 29 to the position shown in FIG. 28, against the rotational bias of the position locks. If the ends of the position locks 528a-b are received within a notch or recess that helps to maintain the locked position, the lock release tool 700 may be used to disengage the position locks from the corresponding notches or recesses. It is contemplated that, in some embodiments, the action of pushing the position locks 528a-b from the positions shown in FIG. 29 to the positions shown in FIG. 28 may be enough to allow the locking mechanism 526 to disengage from the tether 436, for example due to forces from the crimped tether tending to push the locking mechanism 526 toward the unlocked state. However, in other embodiments, crimping tool 600 may be used in combination with lock release tool 700 to transition the locking mechanism 526 from the locked state to the unlocked state.

Referring now to FIG. 33, anchor device 500 is shown with locking mechanism 526 in the locked state. In order to revert the locking mechanism 526 back to the unlocked state, the tip 725 of lock release tool 700 may be inserted through the slot in the tether attachment member 524, until the tip of the tool contacts position lock 528a. Lock release tool 700 may be advanced in direction D to cause position lock 528a to begin rotating against the rotational bias, with the trailing edge of position lock 528a disengaging from contact with the inner surface of the outer perimeter of the tether attachment member 524. Simultaneously, the second member 610 of crimping tool 600 may be inserted into the slot in the tether attachment member 524 and into contact with interior wall 529. The tip end 625 of first member 605 may also be inserted through the slot and into contact with one of the side edges of the locking mechanism 526. Handle end 615 of first member 605 may be rotated in a direction R′ to move the tip ends 625, 630 away from one another, applying a force on locking mechanism 526 to move the locking mechanism toward the unlocked state. The force on crimping tool 600 may be maintained while using the lock release tool 700 to release each of the position locks 528a, 528b. Maintaining force on the crimping tool 600 may help ensure that the position locks 528a, 528b do not revert to the locked position, for example due to their rotational biases, before the locking mechanism 526 is fully transitioned to the unlocked state. With the locking mechanism 526 in the unlocked state, anchor device 500 is free to slide with respect to the tether 436, so that repositioning of the prosthetic heart valve, or re-tensioning of the tether, may be performed.

It should be understood that, although anchor device 500, crimping tool 600, and lock release tool 700 are described for use with a tether formed of a single filament of metal or metal alloy, such as nitinol, these components may function suitably with other types of tethers, including braided fabric or polymer tethers. However, as explained below, a variety of benefits may be obtained from using a nitinol filament tether such as tether 436.

Braided fabric tethers of the prior art may include structures in addition to the fabric to assist in coupling the distal end of the tether to inner frame 250, and to assist in threading a proximal end of the tether through other delivery device components and accessories. For example, the distal end of a typical braided fabric tether may include a metallic plug or other component either inside or outside the fabric tether to assist in coupling the tether to the inner frame 250 and resist being pulled out of the inner frame after coupling. A thin nitinol leader may also be coupled to and extend proximally from the proximal end of the fabric tether, with the nitinol leader helping to thread a proximal end of the tether into delivery devices and related components. These additional components are coupled to (or within) the braided fabric tether at joints, and there may be an increased likelihood of failure at these joints. On the other hand, tether 436 may be formed of a single length of nitinol, and the distal end of the tether may be welded to the inner frame 250, which may reduce the likelihood of failure of the tether 436.

Further, both the cost of manufacturing nitinol tether 436, as well as the time to manufacture the tether and assemble it to the inner frame 250, may be lower than for a typical braided fabric tether. This may be due, at least in part, to the extra steps required to braid the fabric tether, to suture the distal end of the fabric tether to the inner frame 250, and to couple the nitinol leader described above to the fabric tether.

Still further, the delivery of a prosthetic heart valve incorporating a tether and an inner frame similar to inner frame 250 may require or otherwise benefit from the use of a tool to position or re-position the prosthetic heart valve during or immediately after implantation. For example, U.S. Patent Publication No. 2018/0028314, the disclosure of which is hereby incorporated by reference herein, describes an apical positioning tool that can be advanced over the tether and over a portion of the valve stem of the inner frame 250. The valve stem may refer to the tether connection portion 244 and an adjacent portion of struts 243A. As the apical positioning tool passes over the valve stem, a portion of the valve stem may partially collapse, which may provide friction to assist in using the apical positioning tool to reposition the prosthetic heart valve, including rotationally and/or axially. Once a desired position is obtained, the apical positioning tool may be withdrawn from the valve stem, allowing the valve stem to expand, and removed from the body. When inner frame 250 is used with a braided fabric tether, the outer diameter of the valve stem may be between about 0.085 inches (2.159 mm) and about 0.105 inches (2.667 mm), including about 0.095 inches (2.413 mm). This size is due, at least in part, to the relatively large outer diameter of the braided fabric tether. However, as noted above, nitinol tether 436 may have a significantly smaller outer diameter than a braided fabric tether, which may allow the outer diameter of the valve stem to be reduced. The outer diameter of the valve stem could be further reduced due to the method of connecting the nitinol tether to the connection portion 444, which may be via welding instead of a compressive clamping structure, as noted above in connection with FIG. 26. Reducing the outer diameter of the valve stem may allow other accessory tools that need to engage the valve stem, such as an apical positioning tool, to be correspondingly smaller in diameter, which may lead to less trauma to the patient, particularly the portions of the body through which the tool must be inserted (e.g., the chest and/or the ventricular apex).

Yet another benefit of the use of a nitinol tether instead of a braided fabric tether may be found in procedures using a transfemoral delivery route. As noted above, several delivery approaches may be suitable for delivering a tethered prosthetic mitral valve to the native valve annulus. U.S. Patent Publication No. 2016/0324635, the disclosure of which is hereby incorporated by reference herein, describes transfemoral delivery of a tethered mitral valve similar to prosthetic heart valve 300 described above. In transfemoral delivery, the prosthetic valve may be collapsed into a delivery device, and advanced through the femoral vein, into the right atrium, across the atrial septum, and into the left atrium prior to its deployment. In this type of delivery, it may be useful to include a balloon dilator positioned at or toward a distal end of the delivery device. During such a procedure, the balloon may include a lumen in which the tether of the prosthetic heart valve is positioned. The balloon dilator may be susceptible to kinking, particularly as the delivery device traverses tortuous vasculature. If a nitinol tether is coupled to the prosthetic heart valve and within the balloon lumen during delivery, the balloon dilator may be less susceptible to kinking because the nitinol tether provides a more rigid structure within the balloon lumen compared to a less rigid fabric tether.

According to one aspect of the invention, a prosthetic heart valve comprises:

a collapsible and expandable valve frame;

a prosthetic valve assembly disposed within the valve frame;

a tether extending between a first end and a second end, the second end of the tether being coupled to the valve frame, the tether being formed of a metal filament and having a length sufficient to extend through a ventricular wall when the prosthetic heart valve is implanted in an atrioventricular valve annulus; and/or

the metal is a nickel titanium alloy; and/or

a majority of the length of the tether has an outer diameter of between about 0.009 inches and about 0.027 inches; and/or

the majority of the length of the tether has an outer diameter of about 0.018 inches; and/or

the second end of the tether is coupled to the valve frame via welding; and/or

the second end of the tether is coupled to the valve frame via swaging; and/or

the second end of the tether includes a stopper portion having an outer diameter that is larger than an outer diameter of the first end of the tether; and/or

the valve frame includes a plurality of struts that overlie a portion of the tether when the tether is coupled to the valve frame; and/or

the outer diameter of the stopper portion is larger than an inner diameter defined by the plurality of struts overlying the portion of the tether when the tether is coupled to the valve frame.

According to another aspect of the invention, an anchor system for securing a tether of a prosthetic heart valve comprises:

an epicardial anchor having a tether attachment member defining a tether passageway therethrough; and

a locking mechanism positioned within a recess of the tether attachment member, the locking mechanism having a leading tip and being movable from a first position in which the leading tip does not intersect the tether passageway to a second position in which the leading tip intersects the tether passageway, the leading tip of the locking mechanism being configured to frictionally engage the tether, without piercing the tether, when the tether passes through the tether passageway and the locking mechanism is in the second position; and/or

the locking mechanism is substantially triangular; and/or

the locking mechanism includes a trailing edge opposite the leading tip; and/or

a position lock operably coupled to the trailing edge of the locking mechanism, the position lock being rotationally biased to a locked position; and/or

the position lock is rotationally biased via a torsion spring; and/or

when the locking mechanism is in the first position, the position lock is in an unlocked position, and when the locking mechanism is in the second position, the position lock is in the locked position, the rotational bias of the position lock causing the position lock to rotate from the unlocked position toward the locked position as the locking mechanism advances from the first position toward the second position; and/or

the locked position of the position lock, an end of the position lock contacts an inner surface of an outer perimeter of the tether attachment member, the contact between the end of the position lock and the tether attachment member tending to prevent the locking mechanism from moving from the second position toward the first position; and/or

a lock release tool extending between a handle and a tip, the tip of the lock release tool being sized and shaped for insertion through a slot in the tether attachment member and into contact with the position lock to transition the position lock from the locked position to the unlocked position; and/or

the tether attachment member includes an interior wall, the tether passageway being positioned between the interior wall and the locking mechanism; and/or

a crimping tool having first and second members pivotably coupled to one another so that corresponding tips of the first and second members are configured to rotate toward and away from each other; and/or

the first member of the crimping tool is sized and shaped to be inserted through a first slot portion in the tether attachment member to a first use position in contact with the interior wall, and the second member of the crimping tool is sized and shaped to be inserted through a second slot portion in the tether attachment member to a second use position in contact with the locking mechanism, whereupon with the first member of the crimping tool in the first use position and the second member of the crimping tool in the second use position, rotation of the tips of the first and second members toward each other advances the locking mechanism from the first position toward the second position.

While various embodiments have been described above, it should be understood that they have been presented by way of example only, and not limitation. Where methods described above indicate certain events occurring in a certain order, the ordering of certain events may be modified. Additionally, certain of the events may be performed concurrently in a parallel process when possible, as well as being performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a collapsible and expandable valve frame;
a prosthetic valve assembly disposed within the valve frame;
a tether extending between a first end and a second end, the second end of the tether being coupled to the valve frame, the tether being formed of a metal filament and having a length sufficient to extend through a ventricular wall when the prosthetic heart valve is implanted in an atrioventricular valve annulus.

2. The prosthetic heart valve of claim 1, wherein the metal is a nickel titanium alloy.

3. The prosthetic heart valve of claim 1, wherein a majority of the length of the tether has an outer diameter of between about 0.009 inches and about 0.027 inches.

4. The prosthetic heart valve of claim 3, wherein the majority of the length of the tether has an outer diameter of about 0.018 inches.

5. The prosthetic heart valve of claim 1, wherein the second end of the tether is coupled to the valve frame via welding.

6. The prosthetic heart valve of claim 1, wherein the second end of the tether is coupled to the valve frame via swaging.

7. The prosthetic heart valve of claim 1, wherein the second end of the tether includes a stopper portion having an outer diameter that is larger than an outer diameter of the first end of the tether.

8. The prosthetic heart valve of claim 7, wherein the valve frame includes a plurality of struts that overlie a portion of the tether when the tether is coupled to the valve frame.

9. The prosthetic heart valve of claim 8, wherein the outer diameter of the stopper portion is larger than an inner diameter defined by the plurality of struts overlying the portion of the tether when the tether is coupled to the valve frame.

10. An anchor system for securing a tether of a prosthetic heart valve, the anchor system comprising:
an epicardial anchor having a tether attachment member defining a tether passageway therethrough; and
a locking mechanism positioned within a recess of the tether attachment member, the locking mechanism having a leading tip and being movable from a first position in which the leading tip does not intersect the tether passageway to a second position in which the leading tip intersects the tether passageway, the leading tip of the locking mechanism being configured to frictionally engage the tether, without piercing the tether, when the tether passes through the tether passageway and the locking mechanism is in the second position.

11. The anchor system of claim 10, wherein the locking mechanism is substantially triangular.

12. The anchor system of claim 10, wherein the locking mechanism includes a trailing edge opposite the leading tip.

13. The anchor system of claim 12, further comprising a position lock operably coupled to the trailing edge of the locking mechanism, the position lock being rotationally biased to a locked position.

14. The anchor system of claim 13, wherein the position lock is rotationally biased via a torsion spring.

15. The anchor system of claim 13, wherein when the locking mechanism is in the first position, the position lock is in an unlocked position, and when the locking mechanism is in the second position, the position lock is in the locked position, the rotational bias of the position lock causing the position lock to rotate from the unlocked position toward the locked position as the locking mechanism advances from the first position toward the second position.

16. The anchor system of claim 15, wherein in the locked position of the position lock, an end of the position lock contacts an inner surface of an outer perimeter of the tether attachment member, the contact between the end of the position lock and the tether attachment member tending to prevent the locking mechanism from moving from the second position toward the first position.

17. The anchor system of claim 16, further comprising a lock release tool extending between a handle and a tip, the tip of the lock release tool being sized and shaped for insertion through a slot in the tether attachment member and into contact with the position lock to transition the position lock from the locked position to the unlocked position.

18. The anchor system of claim 10, wherein the tether attachment member includes an interior wall, the tether passageway being positioned between the interior wall and the locking mechanism.

19. The anchor system of claim 18, further comprising a crimping tool having first and second members pivotably coupled to one another so that corresponding tips of the first and second members are configured to rotate toward and away from each other.

20. The anchor system of claim 19, wherein the first member of the crimping tool is sized and shaped to be inserted through a first slot portion in the tether attachment member to a first use position in contact with the interior wall, and the second member of the crimping tool is sized and shaped to be inserted through a second slot portion in the tether attachment member to a second use position in contact with the locking mechanism, whereupon with the first member of the crimping tool in the first use position and the second member of the crimping tool in the second use position, rotation of the tips of the first and second members toward each other advances the locking mechanism from the first position toward the second position.
